(19) United States
(12) Patent Application Publication
(10) Pub. No.: US 2024/0065969 A1
(43) Pub. Date: Feb. 29, 2024

(54) OPHTHALMIC TOPICAL CREAM FORMULATIONS AND USES THEREOF

(71) Applicant: Glaukos Corporation, San Clemente, CA (US)

(72) Inventors: Xiaojun Michael Liu, Lake Forest, CA (US); James Shiah, Irvine, CA (US); Tomas Navratil, San Clemente, CA (US); Gabriella Szekely, Irvine, CA (US)

(21) Appl. No.: 18/359,642

(22) Filed: Jul. 26, 2023

Related U.S. Application Data
(60) Provisional application No. 63/392,266, filed on Jul. 26, 2022.

(51) Int. Cl. A61K 9/00; A61K 9/06; A61K 31/4178; A61K 31/522; A61K 31/5575; A61K 31/569; A61K 47/10; A61K 47/14; A61K 47/18; A61K 47/26; A61K 47/32; A61K 47/44

(52) U.S. Cl. CPC A61K 9/0048; A61K 9/06; A61K 31/4178; A61K 31/522; A61K 31/5575; A61K 31/569; A61K 47/10; A61K 47/14; A61K 47/183; A61K 47/26; A61K 47/32; A61K 47/44

(57) ABSTRACT

Described herein are topical cream formulations that include a C₇₋₂₀ alkane, a metal chelating agent, and a buffer.

### RELATED APPLICATIONS

This application claims priority of U.S. Provisional Patent Application No. 63/392,266, filed Jul. 26, 2022, the entire content of which is incorporated herein by reference.

### BACKGROUND

Skin provides many functions, including a barrier or protective function that prevents excessive water loss from internal organs or tissue, and limits entry of toxic, pathogenic, or foreign substances from entering the body. This function, though necessary and beneficial to the subject, creates a significant challenge for topically delivering active pharmaceutical agents (APIs) via formulations such as gels, lotions, creams, ointments, liquids, etc. at therapeutically relevant levels. Thus, there is a continuing need for topical formulations that are physically and chemically stable for, at least, commercial reasons, and are capable of enabling delivery of one or more APIs to a subject in need thereof at therapeutically relevant amounts.

### SUMMARY

Described herein are topical cream formulations, which may be ophthalmic topical cream formulations, that include a C₇₋₂₀ alkane, a metal chelating agent, and/or a buffer. The formulations described herein overcome multiple problems that arise from addressing disease by topical therapeutic formulations. For example, the formulations are chemically and physically stable (e.g., under accelerated storage conditions of 40° C./75 relative humidity (RH)), and the formulations achieve therapeutically relevant bioavailability of active pharmaceutical ingredient in target tissue (e.g., aqueous humor and iris/ciliary body tissues) as observed by preclinical studies.

### BRIEF DESCRIPTION OF THE DRAWINGS

FIG. 1 shows averaged total impurities (%) over time of Formulations 1 and 2 of Example 1: (open square) represents Formulation 1 at pH 3.5 and stored at controlled room temperature; (open circle) represents Formulation 1 at pH 3.5 and stored at 40° C.; (closed square) represents Formulation 2 at pH 5 and stored at controlled room temperature; and (closed circle) represents Formulation 2 at pH 5 and stored at 40° C.

FIG. 2 shows pH changes over time following irradiation of Formulation 5 (comprising Formulation 3 and travoprost) at 25 and 40 kGy eBeam dose (Example 2).

FIG. 3 shows viscosity changes over time following irradiation of Formulation 5 at 25 and 40 kGy eBeam dose (Example 3).

FIG. 4 shows formulation stability changes over time following irradiation of Formulation 5 at 40 kGy eBeam dose (Example 4).

FIG. 5 shows the model drug travoprost concentration in aqueous humor (AH) after a single topical application of Formulation 5 or Formulation 6 (comprising Formulation 4 and travoprost) (Example 6); results are means±standard deviation per time point.

FIG. 6 shows the model drug travoprost concentration in iris/ciliary body (ICB) after a single topical application of Formulation 5 or Formulation 6 (Example 6); results are means±standard deviation per time point.

FIG. 7 shows averaged total impurities (%) over time of Formulations 9 and 10 of Example 1: (open triangle) represents Formulation 10 at pH 5.0 and stored at controlled room temperature; (open circle) represents Formulation 9 at pH 3.5 and stored at controlled room temperature; (closed triangle) represents Formulation 10 at pH 5.0 and stored at 40° C.; and (closed circle) represents Formulation 9 at pH 3.5 and stored at 40° C.

### DETAILED DESCRIPTION

Formulations for topical administration that may include at least one API have been discovered that remain physically and chemically stable over commercially relevant periods of time, which may include at least 6 months or at least one year of storage time. It has been discovered that the formulations enable penetration of one or more APIs into the skin and subsequent delivery of the API to target tissue underlying the skin. It has also been discovered that topical application of the formulations provided herein results in delivery of API through the surface of an eyelid, to an underlying ocular tissue or location. More specifically, it has been found that combinations of isohexadecane, ethylenediaminetetraacetic acid (EDTA) or its salt, and citric acid or its salt in a single topical formulation enable significantly enhanced bioavailability or delivery of one or more APIs to a target site. The formulations are useful in delivering one or more APIs to a target site in a subject, and, by extension, mitigating or treating one or more ocular diseases the subject may be, or may be prone to, suffering from.

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, all numerical values provided herein, unless otherwise indicated, are to be understood as being modified by the term “about.” Accordingly, the last decimal place of a numerical value provided herein indicates its degree of accuracy. Where no other error margins are given, the maximum margin is ascertained by applying the rounding-off convention to the last decimal place or last significant digit when a decimal is not present in the given numerical value.

The terms “alkane,” “alkyl,” or “alkylene” refer to branched, cyclic, or straight chain, or a combination thereof, saturated hydrocarbon or fragment thereof.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The term “Cn-m” refers to a moiety comprising n to m carbon atoms, wherein n and m are integers.

The terms “formulation” and “pharmaceutical formulation” refer to a mixture of at least one compound described herein with a pharmaceutically acceptable carrier. The pharmaceutical formulation facilitates administration of the compound to a patient or subject.

The terms “effective amount” and “therapeutically effective amount” refer to an amount of active ingredient, such as a compound described herein, administered to a subject, either as a single dose or as part of a series of doses, which produces a desired effect. In general, the effective amount can be estimated initially either in cell culture assays or in mammalian animal models, for example, in non-human primates, mice, rabbits, dogs, or pigs. The animal model may also be used to determine the appropriate concentration range and route of administration. Such information can then be used to determine useful doses and routes for administration in non-human subjects and human subjects.

The term “pharmaceutically acceptable carrier” means a pharmaceutically acceptable material, composition, or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical formulations described herein are known in the art and described, for example, in “Remington's Pharmaceutical Sciences” (Genaro (Ed.), Mack Publishing Co., 1985), the entire content of which is incorporated herein by reference.

The term “pharmaceutically acceptable salt” refers to derivatives of the disclosed compounds wherein the parent compound is modified by converting an existing acid or base moiety to its salt form. Pharmaceutically acceptable salts can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two solvents. Lists of suitable salts are found in “Handbook of Pharmaceutical Salts: Properties, Selection, and Use” (P. Henrich Stahl & Camille G. Wermuth (Eds.), VHCA & Wiley-VCH, 2002), the entire content of which is incorporated herein by reference.

The term “refractory disease” refers to a disease that continues to progress during treatment with a pharmaceutical ingredient other than the compounds provided herein, partially responds to the other treatment, or transiently responds to the other treatment. The term may be applied to each of the diseases referred to herein.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the described subject matter and does not pose a limitation on the scope of the subject matter otherwise claimed. No language in the specification should be construed as indicating any non-claimed element essential to practicing the described subject matter.

Use of the term “composition” and “formulation” herein may be interchangeable.

Groupings of alternative elements or embodiments of this disclosure are not to be construed as limitations. Each group member may be referred to and claimed individually or in any combination with other members of the group or other elements found herein. Furthermore, a recited member of a group may be included in, or excluded from, another recited group for reasons of convenience or patentability.

Reference, if any, made to a patent document or other publication in this specification serves as an incorporation herein by reference of the entire content of such document or publication.

Embodiments of this disclosure are illustrative. Accordingly, the present disclosure is not limited to that precisely as shown and described.

### Formulations

In some embodiments, compositions are described including at least one active pharmaceutical ingredient and at least one C₇₋₂₀ alkane.

In other embodiments, compositions can include at least one active pharmaceutical ingredient and at least one metal chelating agent.

In other embodiments, compositions can include at least one active pharmaceutical ingredient and at least one buffer.

In other embodiments, compositions can include at least one active pharmaceutical ingredient and at least one antioxidant.

In other embodiments, compositions can include at least one active pharmaceutical ingredient and at least one base.

In other embodiments, compositions can include at least one active pharmaceutical ingredient and at least one methylxanthine.

In other embodiments, compositions can include at least one active pharmaceutical ingredient and at least one diol.

In other embodiments, compositions can include at least one active pharmaceutical ingredient and at least one emulsifier.

In other embodiments, compositions can include at least one active pharmaceutical ingredient and at least one Lewis acid.

In other embodiments, compositions can include at least one active pharmaceutical ingredient and at least one oil.

In other embodiments, compositions can include at least one active pharmaceutical ingredient and at least one preservative.

In other embodiments, compositions can include at least one active pharmaceutical ingredient and at least one thickening agent.

In other embodiments, compositions can include at least one active pharmaceutical ingredient and at least one tonicity agent.

In some embodiments, compositions are described including at least one active pharmaceutical ingredient, at least one C₇₋₂₀ alkane, and at least one metal chelating agent.

In some embodiments, compositions are described including at least one active pharmaceutical ingredient, at least one C₇₋₂₀ alkane, and at least one buffer.

In some embodiments, compositions are described including at least one active pharmaceutical ingredient, at least one C₇₋₂₀ alkane, and at least one antioxidant.

In some embodiments, compositions are described including at least one active pharmaceutical ingredient, at least one C₇₋₂₀ alkane, and at least one base.

In some embodiments, compositions are described including at least one active pharmaceutical ingredient, at least one C₇₋₂₀ alkane, and at least one methylxanthine.

In some embodiments, compositions are described including at least one active pharmaceutical ingredient, at least one C₇₋₂₀ alkane, and at least one diol.

In some embodiments, compositions are described including at least one active pharmaceutical ingredient, at least one C₇₋₂₀ alkane, and at least one emulsifier.

In some embodiments, compositions are described including at least one active pharmaceutical ingredient, at least one C₇₋₂₀ alkane, and at least one Lewis acid.

In some embodiments, compositions are described including at least one active pharmaceutical ingredient, at least one C₇₋₂₀ alkane, and at least one oil.

In some embodiments, compositions are described including at least one active pharmaceutical ingredient, at least one C₇₋₂₀ alkane, and at least one preservative.

In some embodiments, compositions are described including at least one active pharmaceutical ingredient, at least one C₇₋₂₀ alkane, and at least one thickening agent.

In some embodiments, compositions are described including at least one active pharmaceutical ingredient, at least one C₇₋₂₀ alkane, and at least one tonicity agent.

In some embodiments, compositions are described including at least one active pharmaceutical ingredient, at least one C₇₋₂₀ alkane, at least one metal chelating agent, and at least one buffer.

In some embodiments, compositions are described including at least one active pharmaceutical ingredient, at least one C₇₋₂₀ alkane, at least one metal chelating agent, and at least one antioxidant.

In some embodiments, compositions are described including at least one active pharmaceutical ingredient, at least one C₇₋₂₀ alkane, at least one metal chelating agent, and at least one base.

In some embodiments, compositions are described including at least one active pharmaceutical ingredient, at least one C₇₋₂₀ alkane, at least one metal chelating agent, and at least one methylxanthine.

In some embodiments, compositions are described including at least one active pharmaceutical ingredient, at least one C₇₋₂₀ alkane, at least one metal chelating agent, and at least one diol.

In some embodiments, compositions are described including at least one active pharmaceutical ingredient, at least one C₇₋₂₀ alkane, at least one metal chelating agent, and at least one emulsifier.

In some embodiments, compositions are described including at least one active pharmaceutical ingredient, at least one C₇₋₂₀ alkane, at least one metal chelating agent, and at least one Lewis acid.

In some embodiments, compositions are described including at least one active pharmaceutical ingredient, at least one C₇₋₂₀ alkane, at least one metal chelating agent, and at least one oil.

In some embodiments, compositions are described including at least one active pharmaceutical ingredient, at least one C₇₋₂₀ alkane, at least one metal chelating agent, and at least one preservative.

In some embodiments, compositions are described including at least one active pharmaceutical ingredient, at least one C₇₋₂₀ alkane, at least one metal chelating agent, and at least one thickening agent.

In some embodiments, compositions are described including at least one active pharmaceutical ingredient, at least one C₇₋₂₀ alkane, at least one metal chelating agent, and at least one tonicity agent.

In some embodiments, compositions are described including at least one active pharmaceutical ingredient, at least one C₇₋₂₀ alkane, at least one metal chelating agent, at least one buffer, and at least one antioxidant.

In some embodiments, compositions are described including at least one active pharmaceutical ingredient, at least one C₇₋₂₀ alkane, at least one metal chelating agent, at least one buffer, and at least one base.

In some embodiments, compositions are described including at least one active pharmaceutical ingredient, at least one C₇₋₂₀ alkane, at least one metal chelating agent, at least one buffer, and at least one methylxanthine.

In some embodiments, compositions are described including at least one active pharmaceutical ingredient, at least one C₇₋₂₀ alkane, at least one metal chelating agent, at least one buffer, and at least one diol.

In some embodiments, compositions are described including at least one active pharmaceutical ingredient, at least one C₇₋₂₀ alkane, at least one metal chelating agent, at least one buffer, and at least one emulsifier.

In some embodiments, compositions are described including at least one active pharmaceutical ingredient, at least one C₇₋₂₀ alkane, at least one metal chelating agent, at least one buffer, and at least one Lewis acid.

In some embodiments, compositions are described including at least one active pharmaceutical ingredient, at least one C₇₋₂₀ alkane, at least one metal chelating agent, at least one buffer, and at least one oil.

In some embodiments, compositions are described including at least one active pharmaceutical ingredient, at least one C₇₋₂₀ alkane, at least one metal chelating agent, at least one buffer, and at least one preservative.

In some embodiments, compositions are described including at least one active pharmaceutical ingredient, at least one C₇₋₂₀ alkane, at least one metal chelating agent, at least one buffer, and at least one thickening agent.

In some embodiments, compositions are described including at least one active pharmaceutical ingredient, at least one C₇₋₂₀ alkane, at least one metal chelating agent, at least one buffer, and at least one tonicity agent.

In some embodiments, compositions are described including at least one active pharmaceutical ingredient, at least one C₇₋₂₀ alkane, at least one metal chelating agent, at least one buffer, at least one antioxidant, and at least one base.

In some embodiments, compositions are described including at least one active pharmaceutical ingredient, at least one C₇₋₂₀ alkane, at least one metal chelating agent, at least one buffer, at least one antioxidant, and at least one methylxanthine.

In some embodiments, compositions are described including at least one active pharmaceutical ingredient, at least one C₇₋₂₀ alkane, at least one metal chelating agent, at least one buffer, at least one antioxidant, and at least one diol.

In some embodiments, compositions are described including at least one active pharmaceutical ingredient, at least one C₇₋₂₀ alkane, at least one metal chelating agent, at least one buffer, at least one antioxidant, and at least one emulsifier.

In some embodiments, compositions are described including at least one active pharmaceutical ingredient, at least one C₇₋₂₀ alkane, at least one metal chelating agent, at least one buffer, at least one antioxidant, and at least one Lewis acid.

In some embodiments, compositions are described including at least one active pharmaceutical ingredient, at least one C₇₋₂₀ alkane, at least one metal chelating agent, at least one buffer, at least one antioxidant, and at least one oil.

In some embodiments, compositions are described including at least one active pharmaceutical ingredient, at least one C₇₋₂₀ alkane, at least one metal chelating agent, at least one buffer, at least one antioxidant, and at least one preservative.

In some embodiments, compositions are described including at least one active pharmaceutical ingredient, at least one C₇₋₂₀ alkane, at least one metal chelating agent, at least one buffer, at least one antioxidant, and at least one thickening agent.

In some embodiments, compositions are described including at least one active pharmaceutical ingredient, at least one C₇₋₂₀ alkane, at least one metal chelating agent, at least one buffer, at least one antioxidant, and at least one tonicity agent.

In some embodiments, compositions are described including at least one active pharmaceutical ingredient, at least one C₇₋₂₀ alkane, at least one metal chelating agent, at least one buffer, at least one antioxidant, at least one base, and at least one methylxanthine.

In some embodiments, compositions are described including at least one active pharmaceutical ingredient, at least one C₇₋₂₀ alkane, at least one metal chelating agent, at least one buffer, at least one antioxidant, at least one base, and at least one diol.

In some embodiments, compositions are described including at least one active pharmaceutical ingredient, at least one C₇₋₂₀ alkane, at least one metal chelating agent, at least one buffer, at least one antioxidant, at least one base, and at least one emulsifier.

In some embodiments, compositions are described including at least one active pharmaceutical ingredient, at least one C₇₋₂₀ alkane, at least one metal chelating agent, at least one buffer, at least one antioxidant, at least one base, and at least one Lewis acid.

In some embodiments, compositions are described including at least one active pharmaceutical ingredient, at least one C₇₋₂₀ alkane, at least one metal chelating agent, at least one buffer, at least one antioxidant, at least one base, and at least one oil.

In some embodiments, compositions are described including at least one active pharmaceutical ingredient, at least one C₇₋₂₀ alkane, at least one metal chelating agent, at least one buffer, at least one antioxidant, at least one base, and at least one preservative.

In some embodiments, compositions are described including at least one active pharmaceutical ingredient, at least one C₇₋₂₀ alkane, at least one metal chelating agent, at least one buffer, at least one antioxidant, at least one base, and at least one thickening agent.

In some embodiments, compositions are described including at least one active pharmaceutical ingredient, at least one C₇₋₂₀ alkane, at least one metal chelating agent, at least one buffer, at least one antioxidant, at least one base, and at least one tonicity agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, and at least one diol.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, and at least one emulsifier.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, and at least one Lewis acid.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, and at least one oil.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, and at least one preservative.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, and at least one thickening agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, and at least one tonicity agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, and at least one emulsifier.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, and at least one Lewis acid.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, and at least one oil.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, and at least one preservative.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, and at least one thickening agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, and at least one tonicity agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, and at least one Lewis acid.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, and at least one oil.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, and at least one preservative.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, and at least one thickening agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, and at least one tonicity agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one Lewis acid, and at least one oil.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one Lewis acid, and at least one preservative.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one Lewis acid, and at least one thickening agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one Lewis acid, and at least one tonicity agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one Lewis acid, at least one oil, and at least one preservative.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one Lewis acid, at least one oil, and at least one thickening agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one Lewis acid, at least one oil, and at least one tonicity agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one Lewis acid, at least one oil, at least one preservative, and at least one thickening agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one Lewis acid, at least one oil, at least one preservative, and at least one tonicity agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one Lewis acid, at least one oil, at least one preservative, at least one thickening agent, and at least one tonicity agent.

In some embodiments, compositions are described including at least one active pharmaceutical ingredient, and at least one metal chelating agent.

In some embodiments, compositions are described including at least one active pharmaceutical ingredient, and at least one buffer.

In some embodiments, compositions are described including at least one active pharmaceutical ingredient, and at least one antioxidant.

In some embodiments, compositions are described including at least one active pharmaceutical ingredient, and at least one base.

In some embodiments, compositions are described including at least one active pharmaceutical ingredient, and at least one methylxanthine.

In some embodiments, compositions are described including at least one active pharmaceutical ingredient, and at least one diol.

In some embodiments, compositions are described including at least one active pharmaceutical ingredient, and at least one emulsifier.

In some embodiments, compositions are described including at least one active pharmaceutical ingredient, and at least one Lewis acid.

In some embodiments, compositions are described including at least one active pharmaceutical ingredient, and at least one oil.

In some embodiments, compositions are described including at least one active pharmaceutical ingredient, and at least one preservative.

In some embodiments, compositions are described including at least one active pharmaceutical ingredient, and at least one thickening agent.

In some embodiments, compositions are described including at least one active pharmaceutical ingredient, and at least one tonicity agent.

In some embodiments, compositions are described including at least one active pharmaceutical ingredient, at least one metal chelating agent, and at least one buffer.

In some embodiments, compositions are described including at least one active pharmaceutical ingredient, at least one metal chelating agent, and at least one antioxidant.

In some embodiments, compositions are described including at least one active pharmaceutical ingredient, at least one metal chelating agent, and at least one base.

In some embodiments, compositions are described including at least one active pharmaceutical ingredient, at least one metal chelating agent, and at least one methylxanthine.

In some embodiments, compositions are described including at least one active pharmaceutical ingredient, at least one metal chelating agent, and at least one diol.

In some embodiments, compositions are described including at least one active pharmaceutical ingredient, at least one metal chelating agent, and at least one emulsifier.

In some embodiments, compositions are described including at least one active pharmaceutical ingredient, at least one metal chelating agent, and at least one Lewis acid.

In some embodiments, compositions are described including at least one active pharmaceutical ingredient, at least one metal chelating agent, and at least one oil.

In some embodiments, compositions are described including at least one active pharmaceutical ingredient, at least one metal chelating agent, and at least one preservative.

In some embodiments, compositions are described including at least one active pharmaceutical ingredient, at least one metal chelating agent, and at least one thickening agent.

In some embodiments, compositions are described including at least one active pharmaceutical ingredient, at least one metal chelating agent, and at least one tonicity agent.

In some embodiments, compositions are described including at least one active pharmaceutical ingredient, at least one metal chelating agent, at least one buffer, and at least one antioxidant.

In some embodiments, compositions are described including at least one active pharmaceutical ingredient, at least one metal chelating agent, at least one buffer, and at least one base.

In some embodiments, compositions are described including at least one active pharmaceutical ingredient, at least one metal chelating agent, at least one buffer, and at least one methylxanthine.

In some embodiments, compositions are described including at least one active pharmaceutical ingredient, at least one metal chelating agent, at least one buffer, and at least one diol.

In some embodiments, compositions are described including at least one active pharmaceutical ingredient, at least one metal chelating agent, at least one buffer, and at least one emulsifier.

In some embodiments, compositions are described including at least one active pharmaceutical ingredient, at least one metal chelating agent, at least one buffer, and at least one Lewis acid.

In some embodiments, compositions are described including at least one active pharmaceutical ingredient, at least one metal chelating agent, at least one buffer, and at least one oil.

In some embodiments, compositions are described including at least one active pharmaceutical ingredient, at least one metal chelating agent, at least one buffer, and at least one preservative.

In some embodiments, compositions are described including at least one active pharmaceutical ingredient, at least one metal chelating agent, at least one buffer, and at least one thickening agent.

In some embodiments, compositions are described including at least one active pharmaceutical ingredient, at least one metal chelating agent, at least one buffer, and at least one tonicity agent.

In some embodiments, compositions are described including at least one active pharmaceutical ingredient, at least one metal chelating agent, at least one buffer, at least one antioxidant, and at least one base.

In some embodiments, compositions are described including at least one active pharmaceutical ingredient, at least one metal chelating agent, at least one buffer, at least one antioxidant, and at least one methylxanthine.

In some embodiments, compositions are described including at least one active pharmaceutical ingredient, at least one metal chelating agent, at least one buffer, at least one antioxidant, and at least one diol.

In some embodiments, compositions are described including at least one active pharmaceutical ingredient, at least one metal chelating agent, at least one buffer, at least one antioxidant, and at least one emulsifier.

In some embodiments, compositions are described including at least one active pharmaceutical ingredient, at least one metal chelating agent, at least one buffer, at least one antioxidant, and at least one Lewis acid.

In some embodiments, compositions are described including at least one active pharmaceutical ingredient, at least one metal chelating agent, at least one buffer, at least one antioxidant, and at least one oil.

In some embodiments, compositions are described including at least one active pharmaceutical ingredient, at least one metal chelating agent, at least one buffer, at least one antioxidant, and at least one preservative.

In some embodiments, compositions are described including at least one active pharmaceutical ingredient, at least one metal chelating agent, at least one buffer, at least one antioxidant, and at least one thickening agent.

In some embodiments, compositions are described including at least one active pharmaceutical ingredient, at least one metal chelating agent, at least one buffer, at least one antioxidant, and at least one tonicity agent.

In some embodiments, compositions are described including at least one active pharmaceutical ingredient, at least one metal chelating agent, at least one buffer, at least one antioxidant, at least one base, and at least one methylxanthine.

In some embodiments, compositions are described including at least one active pharmaceutical ingredient, at least one metal chelating agent, at least one buffer, at least one antioxidant, at least one base, and at least one diol.

In some embodiments, compositions are described including at least one active pharmaceutical ingredient, at least one metal chelating agent, at least one buffer, at least one antioxidant, at least one base, and at least one emulsifier.

In some embodiments, compositions are described including at least one active pharmaceutical ingredient, at least one metal chelating agent, at least one buffer, at least one antioxidant, at least one base, and at least one Lewis acid.

In some embodiments, compositions are described including at least one active pharmaceutical ingredient, at least one metal chelating agent, at least one buffer, at least one antioxidant, at least one base, and at least one oil.

In some embodiments, compositions are described including at least one active pharmaceutical ingredient, at least one metal chelating agent, at least one buffer, at least one antioxidant, at least one base, and at least one preservative.

In some embodiments, compositions are described including at least one active pharmaceutical ingredient, at least one metal chelating agent, at least one buffer, at least one antioxidant, at least one base, and at least one thickening agent.

In some embodiments, compositions are described including at least one active pharmaceutical ingredient, at least one metal chelating agent, at least one buffer, at least one antioxidant, at least one base, and at least one tonicity agent.

In some embodiments, compositions are described including at least one active pharmaceutical ingredient, at least one metal chelating agent, at least one buffer, at least one antioxidant, at least one base, at least one methylxanthine, and at least one diol.

In some embodiments, compositions are described including at least one active pharmaceutical ingredient, at least one metal chelating agent, at least one buffer, at least one antioxidant, at least one base, at least one methylxanthine, and at least one emulsifier.

In some embodiments, compositions are described including at least one active pharmaceutical ingredient, at least one metal chelating agent, at least one buffer, at least one antioxidant, at least one base, at least one methylxanthine, and at least one Lewis acid.

In some embodiments, compositions are described including at least one active pharmaceutical ingredient, at least one metal chelating agent, at least one buffer, at least one antioxidant, at least one base, at least one methylxanthine, and at least one oil.

In some embodiments, compositions are described including at least one active pharmaceutical ingredient, at least one metal chelating agent, at least one buffer, at least one antioxidant, at least one base, at least one methylxanthine, and at least one preservative.

In some embodiments, compositions are described including at least one active pharmaceutical ingredient, at least one metal chelating agent, at least one buffer, at least one antioxidant, at least one base, at least one methylxanthine, and at least one thickening agent.

In some embodiments, compositions are described including at least one active pharmaceutical ingredient, at least one metal chelating agent, at least one buffer, at least one antioxidant, at least one base, at least one methylxanthine, and at least one tonicity agent.

In some embodiments, compositions are described including at least one active pharmaceutical ingredient, at least one metal chelating agent, at least one buffer, at least one antioxidant, at least one base, at least one methylxanthine, at least one diol, and at least one emulsifier.

In some embodiments, compositions are described including at least one active pharmaceutical ingredient, at least one metal chelating agent, at least one buffer, at least one antioxidant, at least one base, at least one methylxanthine, at least one diol, and at least one Lewis acid.

In some embodiments, compositions are described including at least one active pharmaceutical ingredient, at least one metal chelating agent, at least one buffer, at least one antioxidant, at least one base, at least one methylxanthine, at least one diol, and at least one oil.

In some embodiments, compositions are described including at least one active pharmaceutical ingredient, at least one metal chelating agent, at least one buffer, at least one antioxidant, at least one base, at least one methylxanthine, at least one diol, and at least one preservative.

In some embodiments, compositions are described including at least one active pharmaceutical ingredient, at least one metal chelating agent, at least one buffer, at least one antioxidant, at least one base, at least one methylxanthine, at least one diol, and at least one thickening agent.

In some embodiments, compositions are described including at least one active pharmaceutical ingredient, at least one metal chelating agent, at least one buffer, at least one antioxidant, at least one base, at least one methylxanthine, at least one diol, and at least one tonicity agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, and at least one Lewis acid.

In some embodiments, compositions are described including at least one active pharmaceutical ingredient, at least one metal chelating agent, at least one buffer, at least one antioxidant, at least one base, at least one methylxanthine, at least one diol, at least one emulsifier, and at least one oil.

In some embodiments, compositions are described including at least one active pharmaceutical ingredient, at least one metal chelating agent, at least one buffer, at least one antioxidant, at least one base, at least one methylxanthine, at least one diol, at least one emulsifier, and at least one preservative.

In some embodiments, compositions are described including at least one active pharmaceutical ingredient, at least one metal chelating agent, at least one buffer, at least one antioxidant, at least one base, at least one methylxanthine, at least one diol, at least one emulsifier, and at least one thickening agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, and at least one tonicity agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one Lewis acid, and at least one oil.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one Lewis acid, and at least one preservative.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one Lewis acid, and at least one thickening agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one Lewis acid, and at least one tonicity agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one Lewis acid, at least one oil, and at least one preservative.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one Lewis acid, at least one oil, and at least one thickening agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one Lewis acid, at least one oil, and at least one tonicity agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one Lewis acid, at least one oil, at least one preservative, and at least one thickening agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one Lewis acid, at least one oil, at least one preservative, and at least one tonicity agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one Lewis acid, at least one oil, at least one preservative, at least one thickening agent, and at least one tonicity agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, and at least one oil.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, and at least one preservative.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, and at least one thickening agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, and at least one tonicity agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one oil, and at least one preservative.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one oil, and at least one thickening agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one oil, and at least one tonicity agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one oil, at least one preservative, and at least one thickening agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one oil, at least one preservative, and at least one tonicity agent.

In some embodiments, compositions are described including at least one active pharmaceutical ingredient, at least one C₇₋₂₀ alkane, at least one metal chelating agent, at least one buffer, at least one antioxidant, at least one base, at least one methylxanthine, at least one diol, at least one emulsifier, at least one oil, at least one preservative, at least one thickening agent, and at least one tonicity agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, and at least one oil.

In some embodiments, compositions are described including at least one active pharmaceutical ingredient, at least one metal chelating agent, at least one buffer, at least one antioxidant, at least one base, at least one methylxanthine, at least one diol, at least one emulsifier, and at least one preservative.

In some embodiments, compositions are described including at least one active pharmaceutical ingredient, at least one metal chelating agent, at least one buffer, at least one antioxidant, at least one base, at least one methylxanthine, at least one diol, at least one emulsifier, and at least one thickening agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, and at least one tonicity agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one oil, and at least one preservative.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one oil, and at least one thickening agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one oil, and at least one tonicity agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one oil, at least one preservative, and at least one thickening agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one oil, at least one preservative, and at least one tonicity agent.

In some embodiments, compositions are described including at least one active pharmaceutical ingredient, at least one metal chelating agent, at least one buffer, at least one antioxidant, at least one base, at least one methylxanthine, at least one diol, at least one emulsifier, at least one oil, at least one preservative, at least one thickening agent, and at least one tonicity agent.

In some embodiments, described herein are topical formulations that include a C₇₋₂₀ or C₁₀₋₂₀ alkane (e.g., isohexadecane), a metal chelating agent (e.g., EDTA or a salt thereof), and a buffer (e.g., acetic acid, ascorbic acid, azeliac acid, boric acid, carbonic acid, citric acid, glycolic acid, lactic acid, phosphoric acid, or a salt thereof). The formulations may include about 0.05% to about 3% w/w C₇₋₂₀ or C₁₀₋₂₀ alkane, about 0.005% to about 0.5% w/w metal chelating agent, and about 1 mM to about 500 mM buffer. In some embodiments, the formulations may include about 0.1% to about 2%, e.g., about 0.3% to about 1%, C₇₋₂₀ or C₁₀₋₂₀ alkane. In some embodiments, the formulations may include about 0.05% to about 0.3% w/w, e.g., about 0.1% to about 0.25%, metal chelating agent. In some embodiments, the formulations may include about 5 mM to about 100 mM, e.g., about 5 mM to about 25 mM, buffer. The formulations may further include one or more of the following:
a. at least one antioxidant (e.g., butylated hydroxyanisole (BHA), butylated hydroxytoluene (BHT), vitamin A, vitamin B₁, vitamin B₂, vitamin B₃, vitamin B₅, vitamin B₆, vitamin B₉, vitamin B₁₂, vitamin C, vitamin D, vitamin E, or vitamin K), which in some embodiments may be about 0.01 to about 0.5% w/w of the formulation;
b. at least one base (e.g., KOH, NaOH, or NH₄OH), which may be used to adjust pH as needed;
c. at least one methylxanthine (e.g., aminophylline, caffeine, 3-isobutyl-1-methylxanthine (IBMX), paraxanthine, pentoxifylline, theobromine, or theophylline), which in some embodiments may be about 0.05 to about 0.5% w/w of the formulation;
d. at least one diol (e.g., a polyalkylene diol (e.g., polyethylene glycol, propylene glycol, or both)), which in some embodiments may be about 0.1 to about 10% w/w of the formulation;
e. at least one emulsifier (e.g., cetyl alcohol, lecithin, polysorbate 20, polysorbate 40, polysorbate 60, or polysorbate 80), which in some embodiments may be about 0.02 to about 5% w/w of the formulation;
f. at least one Lewis acid (e.g., MgCl₂ or CaCl₂)), which in some embodiments may be about 0.01 to about 3% w/w of the formulation;
g. at least one oil (e.g., castor oil or mineral oil), which in some embodiments may be about 0.1 to about 25% w/w of the formulation;
h. at least one preservative (e.g., benzalkonium chloride (BAK), benzododecinium bromide, a biguanide compound (e.g., a bisbiguanide (e.g., alexidine), buformin, chlorproguanil, metformin, phenformin, polyhexamethylene biguanide (PHMB), polyhydroxy methyl biguanide, or proguanil), chlorobutanol, a polyquaternium (e.g., polyquaternium-1), a paraben (e.g., butylparaben, ethylparaben, methylparaben, or propylparaben), phenoxyethanol, sodium chlorite, sodium chlorite combined with zinc chloride, SOFZIA (e.g., an ionic buffer containing borate, sorbitol, propylene glycol, and zinc), or thimerosal), which in some embodiments may be about 0.00001 to about 0.3% w/w of the formulation;
i. at least one thickening agent (e.g., a carbomer, carboxymethyl cellulose (CMC), carrageenan, crosslinked CMC, crosslinked hyaluronic acid, hyaluronic acid, polyvinyl pyrrolidone (PVP), acrylamide/sodium acryloyldimethyl taurate copolymer, or xanthan gum), which in some embodiments may be about 0.01 to about 10% w/w of the formulation;
j. at least one tonicity agent (e.g., sorbitol), which in some embodiments may be about 0.5 to about 10% w/w of the formulation; or
k. water, which in some embodiments may be about 1 to about 90% w/w of the formulation.

In some embodiments, an API in the formulation may have anti-microbial properties, and therefore a separate preservative may be optional. Accordingly, in some embodiments, the formulations provided herein include a preservative that is not the API. In other embodiments, the formulations provided herein do not include a preservative. In some embodiments, the formulations do not include benzalkonium chloride.

In some embodiments, the formulations provided herein include a methylxanthine (e.g., caffeine). In other embodiments, the formulations provided herein do not include a methylxanthine (e.g., caffeine).

In some embodiments, the formulations provided herein include acrylamide/sodium acryloyldimethyl taurate copolymer, citric acid or a salt thereof, EDTA or a salt thereof, isohexadecane, and polysorbate 80. In some embodiments, the formulations provided herein include acrylamide/sodium acryloyldimethyl taurate copolymer, caffeine, citric acid or a salt thereof, EDTA or a salt thereof, isohexadecane, and polysorbate 80.

At least one API may be included in the formulations provided herein. The API may have a molar mass of about 1,500 g/mol or less, e.g., about 1,000 g/mol or less, e.g., about 500 g/mol or less. In some embodiments, the API is amphiphilic, hydrophilic, or hydrophobic. In some embodiments, the API is aflibercept, apraclonidine, atropine, betaxolol, brimonidine, brinzolamide, carbachol, dexamethasone, dipivefrin, donepezil, dorzolamide, levobunalol, metipranolol, pilocarpine, physostigmine, a prostaglandin (e.g. bimatoprost, latanoprost, prostaglandin F2a as an isopropyl ester, tafluprost, or travoprost, or their corresponding free acid), a steroid (e.g., an androgen, a corticosteroid (e.g., a glucocorticoid or a mineralocorticoid (e.g., loteprednol (loteprednol etabonate))), an estrogen, or a progestogen), or timolol, or a pharmaceutically acceptable salt thereof.

In some embodiments, formulations are described that include zero or up to about 0.16% w/w caffeine, about 0.5 to about 4% w/w carbomer homopolymer type C, about 0.5% w/w cetyl alcohol, about 0.02 to about 0.2% w/w disodium EDTA, about 0.05 to about 0.18 methyl paraben, about 10% w/w mineral oil, about 2% PEG 8000, about 5% w/w polyoxyl 35 castor oil, about 2% w/w polysorbate 80, about 0.01 to about 0.02 propyl paraben, about 4.4% w/w propylene glycol, zero or up to about 4% w/w Sepineo P600 (the Sepineo P600 comprising about 30-40% w/w acrylamide/sodium acryloyldimethyl taurate copolymer (e.g., with an ultra-high molecular weight of at least 10M Da), about 5-10% w/w polysorbate 80 (in addition to other polysorbate 80 present), and about 20-25% w/w isohexadecane), about 6% w/w sorbitol, and/or about 0.004 to about 4% w/w of an active pharmaceutical ingredient.

In some embodiments, the herein described formulations do not include caffeine.

In some embodiments, the herein described formulations do not include Sepineo P600, but may include one or more components of Sepineo P600.

In some embodiments, described herein are compositions, comprising about 0.05% to about 3% w/w C₇₋₂₀ alkane, about 0.005% to about 0.5% w/w metal chelating agent, and about 1 mM to about 500 mM buffer. In some embodiments, the compositions may include one or more of the following:
a. at least one antioxidant;
b. at least one base;
c. at least one methylxanthine;
d. at least one diol;
e. at least one emulsifier;
f. at least one Lewis acid;
g. at least one oil;
h. at least one preservative;
i. at least one thickening agent;
j. at least one tonicity agent;
k. water; or
l. at least one active pharmaceutical ingredient.

In some embodiments, described herein are compositions, including:
zero or up to about 0.16% w/w caffeine;
about 0.5 to about 4% w/w carbomer homopolymer type C;
about 0.5% w/w cetyl alcohol;
about 0.02 to about 0.2% w/w disodium EDTA;
about 0.05 to about 0.18 methyl paraben;
about 10% w/w mineral oil;
about 2% PEG 8000;
about 5% w/w polyoxyl 35 castor oil;
about 2% w/w of a first polysorbate 80;
about 0.01 to about 0.02 propyl paraben;
about 4.4% w/w propylene glycol;
zero or up to about 4% w/w of a component, the component comprising about 30-40% w/w acrylamide/sodium acryloyldimethyl taurate copolymer, about 5-10% w/w of a second polysorbate 80, and about 20-25% w/w isohexadecane;
about 6% w/w sorbitol;
about 0.004 to about 4% w/w of at least one active pharmaceutical ingredient; and
a pH of about 3.5±0.5 or about 5.5±0.5;
wherein the composition optionally includes about 0.025% w/w butylated hydroxyanisole, and about 6 mM to about 7 mM potassium citrate.

In some embodiments, described herein are compositions, including:
zero or up to about 0.16% w/w caffeine;
about 0.5 to about 4% w/w carbomer homopolymer type C;
about 0.5% w/w cetyl alcohol;
about 0.02 to about 0.2% w/w disodium EDTA;
about 0.05 to about 0.18 methyl paraben;
about 10% w/w mineral oil;
about 2% PEG 8000;
about 5% w/w polyoxyl 35 castor oil;
about 2% w/w of a first polysorbate 80;
about 0.01 to about 0.02 propyl paraben;
about 4.4% w/w propylene glycol;
zero or up to about 4% w/w of a component, the component comprising about 30-40% w/w acrylamide/sodium acryloyldimethyl taurate copolymer, about 5-10% w/w of a second polysorbate 80, and about 20-25% w/w isohexadecane;
about 6% w/w sorbitol;
about 0.004 to about 4% w/w of at least one active pharmaceutical ingredient; and
a pH of about 3.5±0.5 or about 5.5±0.5;
wherein the composition includes about 0.025% w/w butylated hydroxyanisole, and about 6 mM to about 7 mM potassium citrate.

In some embodiments, described herein are compositions, including:
zero or up to about 0.16% w/w caffeine;
about 0.5 to about 4% w/w carbomer homopolymer type C;
about 0.5% w/w cetyl alcohol;
about 0.02 to about 0.2% w/w disodium EDTA;
about 0.05 to about 0.18 methyl paraben;
about 10% w/w mineral oil;
about 2% PEG 8000;
about 5% w/w polyoxyl 35 castor oil;
about 2% w/w of a first polysorbate 80;
about 0.01 to about 0.02 propyl paraben;
about 4.4% w/w propylene glycol;
zero or up to about 4% w/w of a component, the component comprising about 30-40% w/w acrylamide/sodium acryloyldimethyl taurate copolymer, about 5-10% w/w of a second polysorbate 80, and about 20-25% w/w isohexadecane;
about 6% w/w sorbitol;
about 0.004 to about 4% w/w of at least one active pharmaceutical ingredient; and
a pH of about 3.5±0.5 or about 5.5±0.5;
wherein the composition does not include butylated hydroxyanisole and does not include potassium citrate.

In some embodiments, described herein are compositions, including:
zero or up to about 0.16% w/w caffeine;
about 0.5 to about 4% w/w carbomer homopolymer type C;
about 0.5% w/w cetyl alcohol;
about 0.02 to about 0.2% w/w disodium EDTA;
about 0.05 to about 0.18 methyl paraben;
about 10% w/w mineral oil;
about 2% PEG 8000;
about 5% w/w polyoxyl 35 castor oil;
about 2% w/w of a first polysorbate 80;
about 0.01 to about 0.02 propyl paraben;
about 4.4% w/w propylene glycol;
zero or up to about 4% w/w of a component, the component comprising about 30-40% w/w acrylamide/sodium acryloyldimethyl taurate copolymer, about 5-10% w/w of a second polysorbate 80, and about 20-25% w/w isohexadecane;
about 6% w/w sorbitol;
about 0.004 to about 4% w/w of at least one active pharmaceutical ingredient; and
a pH of about 3.5±0.5 or about 5.5±0.5;
wherein the composition does not include butylated hydroxyanisole.

In some embodiments, described herein are compositions, including:
zero or up to about 0.16% w/w caffeine;
about 0.5 to about 4% w/w carbomer homopolymer type C;
about 0.5% w/w cetyl alcohol;
about 0.02 to about 0.2% w/w disodium EDTA;
about 0.05 to about 0.18 methyl paraben;
about 10% w/w mineral oil;
about 2% PEG 8000;
about 5% w/w polyoxyl 35 castor oil;
about 2% w/w of a first polysorbate 80;
about 0.01 to about 0.02 propyl paraben;
about 4.4% w/w propylene glycol;
zero or up to about 4% w/w of a component, the component comprising about 30-40% w/w acrylamide/sodium acryloyldimethyl taurate copolymer, about 5-10% w/w of a second polysorbate 80, and about 20-25% w/w isohexadecane;
about 6% w/w sorbitol;
about 0.004 to about 4% w/w of at least one active pharmaceutical ingredient; and
a pH of about 3.5±0.5 or about 5.5±0.5;
wherein the composition does not include potassium citrate.

In some embodiments, described herein are compositions which are cream formulations.

In some embodiments, described herein are compositions which are oil-in-water cream formulations.

In some embodiments, described herein are compositions, wherein the C₇₋₂₀ alkane is an emollient.

In some embodiments, described herein are compositions, wherein the C₇₋₂₀ alkane is isohexadecane.

In some embodiments, described herein are compositions, including the at least one active pharmaceutical ingredient, which is travoprost.

In some embodiments, described herein are compositions, wherein the metal chelating agent is an ethylenediaminetetraacetic acid or a salt thereof.

In some embodiments, described herein are compositions, wherein the buffer is a citric acid or a salt thereof.

In some embodiments, described herein are compositions, including the at least one active pharmaceutical ingredient, which is selected from aflibercept, apraclonidine, atropine, betaxolol, brimonidine, brinzolamide, carbachol, dexamethasone, dipivefrin, donepezil, dorzolamide, levobunalol, metipranolol, pilocarpine, physostigmine, a prostaglandin, a steroid, or timolol, or a pharmaceutically acceptable salt thereof.

In some embodiments, described herein are compositions, including the at least one active pharmaceutical ingredient, which is selected from:
a. a prostaglandin selected from bimatoprost, latanoprost, prostaglandin F2a as an isopropyl ester, tafluprost, or travoprost, or their corresponding free acid; or
b. a steroid selected from an androgen, a corticosteroid, an estrogen, or a progestogen.

In some embodiments, described herein are compositions, including the at least one active pharmaceutical ingredient, which is selected from a glucocorticoid or a mineralocorticoid.

In some embodiments, described herein are compositions, including the at least one active pharmaceutical ingredient, which is loteprednol etabonate.

In some embodiments, described herein are compositions, wherein the C₇₋₂₀ alkane is isohexadecane, the metal chelating agent is ethylenediaminetetraacetic acid disodium salt, and the buffer is potassium citrate.

In some embodiments, described herein are compositions, comprising about 0.4-0.5% w/w isohexadecane, and about 0.012% w/w travoprost.

In some embodiments, described herein are compositions, comprising about 1% , about 1.25%, about 1.5%, about 2%, about 3%, or about 4% w/w pilocarpine (as the neutral free base), pilocarpine nitrate, or pilocarpine hydrochloride.

In some embodiments, described herein are compositions, comprising about 6-7 mM potassium citrate, about 0.2% w/w ethylenediaminetetraacetic acid disodium salt, about 0.4-0.5% w/w isohexadecane, and about 0.012% w/w travoprost.

The formulations herein may be applied topically to a portion of a subject's skin. In some embodiments, the topical application is to an eyelid (e.g., an upper eyelid or a lower eyelid, a combination thereof on one or both eyes of the subject). The formulations provided herein allow for the API contained therein to penetrate the skin, and thereby deliver the API to a tissue of the subject, including an organ or an internal tissue or region of the subject. In some embodiments, the formulations herein deliver an API therein to an eye of the subject following application of the formulation to the subject's skin.

A number of factors need to be balanced when developing a topical formulation, including controlling the formation of API side products (impurities), the physical appearance of the formulation such as homogeneity and viscosity, as well as the subject's somatosensory experience (e.g., mechano-sensation and thermo-sensation) when using the formulation.

APIs may degrade at varying rates as a result of various factors related to their immediate chemical environment. For example, pilocarpine includes a lactone ring that may undergo hydrolysis. The hydrolysis of the lactone ring of pilocarpine can be catalyzed by both acid and base and can be controlled by pH. Acid-catalyzed hydrolysis is slower. Without being bound by theory, acids catalyze the reaction making the carbonyl carbon more partially positive, by protonation of the carbonyl oxygen and therefore more susceptible for nucleophilic attack. Base catalyzed hydrolysis is faster and generally not reversible. For example, without being bound by theory, a strong base, such as NaOH, can hydrolyze the lactone ring of pilocarpine, which results in saponification to a pilocarpine acid salt. Once saponification occurs, this reaction is generally not reversible. To minimize this chemical degradation, the pH should be maintained below the pKa of pilocarpine (˜pH 6.5), and preferably lower than pH 5.0 and below, or even lower than pH 4.0. A Lewis acid (electron-pair acceptor), such as MgCl₂ or CaCl₂), may be used to promote the re-closure of a hydrolyzed lactone ring, by stabilizing the electrophilic carbonyl-carbon and therefore minimize the formulation of side product by the nucleophilic attack on carbonyl bond. For example, MgCl₂ is used to promote the re-closure of lactone ring and prevent pilocarpine from hydrolyzing to pilocarpine acid. Surprisingly, pilocarpine-containing formulations have been discovered that have a pH of about 3.5, the formulations do not include a Lewis acid, such as MgCl₂ or CaCl₂), or a buffer (e.g., one having a pKa in a range relevant for a formulation having a pH of about 3.5), and yet the pilocarpine formulations remain chemically stable for at least about 6 months under accelerated storage conditions of 40° C., which corresponds to commercially relevant storage conditions.

The physical appearance of topical formulations such as homogeneity and viscosity are two factors to be considered during formulation development. A formulation thickener, such as CARBOPOL(R) if included in a formulation, does not achieve desirable physical appearance or viscosity when the carboxylic acid groups of its acrylates are protonated under acidic conditions. To achieve desirable formulation viscosity, the pH is controlled above pH around 3, and preferably pH around 4.0 and above, or more preferably pH around 5.0 and above. In some embodiments, formulations provided herein may have a pH of about 3.5±0.5, about 4.5±0.5, or about 5.5±0.5. For example, Formulation 1 and 9 have a pH of about 3.5, whereas Formulations 3, 4, 5, 6, 7, and 8 have a pH of about 5.5.

In some embodiments, the formulation is Formulation 3, shown in Table 1, which may include an API. In some embodiments, the formulation comprises Formulation 3, 1-500 mM potassium citrate buffer, and an API. In some embodiments, the formulation comprises Formulation 3, about 1-500 mM potassium citrate buffer, and travoprost. In some embodiments, the formulation comprises Formulation 3, about 10 mM potassium citrate buffer, and about 0.004 to about 0.012% w/w travoprost (e.g., about 0.004, about 0.008, or about 0.012% w/w travoprost).

TABLE 1
Formulation 3 and Comparative Formulation 4.

| | Formulation 3 | Formulation 4 |
|---|---|---|
| Ingredients | (% w/w) | (% w/w) |
| 10 mM Potassium citrate buffer | 66.823 | — |
| solution/Water | | |
| Water | — | 65.123 |
| Disodium EDTA | 0.2 | 0.02 |
| Polysorbate 80 | 2.0 | 2.0 |
| Caffeine | 0.16 | 0.16 |
| Methyl Paraben | 0.05 | 0.05 |
| Sorbitol | 6.0 | 6.0 |
| Sepineo P600 | 2.0 | 0.0 |
| (comprising 20-25% | | |
| isohexadecane) | | |
| Carbomer Homopolymer Type C | 0.5 | 1.5 |
| Mineral Oil | 10.0 | 10.0 |
| Poly-Oxyl 35 Castor Oil | 5.0 | 5.0 |
| Cetyl Alcohol | 0.5 | 0.5 |
| PEG 8000 | 2.0 | 2.0 |
| Propyl Paraben | 0.01 | 0.01 |
| Propylene Glycol | 4.4 | 4.4 |
| BHA | 0.025 | 0.025 |
| NaOH 25% w/w solution | pH adjuster | pH adjuster |

In some embodiments, the formulation provided herein comprises isohexadecane and travoprost. In some embodiments, the formulation includes isohexadecane, travoprost, an ethylenediaminetetraacetic acid (e.g., disodium EDTA), and a citrate buffer (e.g., a potassium citrate buffer). In some embodiments, the formulation is Formulation 5, Formulation 7, or Formulation 8, shown in Table 2.

TABLE 2
Formulation 5, Formulation 7, Formulation
8, and Comparative Formulation 6.

| | Formulation | Formulation | Formulation | Formulation |
|---|---|---|---|---|
| | 5 | 6 | 7 | 8 |
| Ingredients | (% w/w) | (% w/w) | (% w/w) | (% w/w) |
| 10 mM | 66.823 | — | 66.831 | 66.827 |
| Potassium | | | | |
| citrate buffer | | | | |
| solution | | | | |
| in water | | | | |
| Water | — | 65.123 | — | — |
| Travoprost | 0.012 | 0.012 | 0.004 | 0.008 |
| Disodium | 0.2 | 0.02 | 0.2 | 0.2 |
| EDTA | | | | |
| Polysorbate 80 | 2.0 | 2.0 | 2.0 | 2.0 |
| Caffeine | 0.16 | 0.16 | 0.16 | 0.16 |
| Methyl Paraben | 0.05 | 0.05 | 0.05 | 0.05 |
| Sorbitol | 6.0 | 6.0 | 6.0 | 6.0 |
| Sepineo P600 | 2.0 | 0.0 | 2.0 | 2.0 |
| (comprising | | | | |
| 20-25% | | | | |
| isohexadecane) | | | | |
| Carbomer | 0.5 | 1.5 | 0.5 | 0.5 |
| Homopolymer | | | | |
| Type C | | | | |
| Mineral Oil | 10.0 | 10.0 | 10.0 | 10.0 |
| Poly-Oxyl 35 | 5.0 | 5.0 | 5.0 | 5.0 |
| Castor Oil | | | | |
| Cetyl Alcohol | 0.5 | 0.5 | 0.5 | 0.5 |
| PEG 8000 | 2.0 | 2.0 | 2.0 | 2.0 |
| Propyl | 0.01 | 0.01 | 0.01 | 0.01 |
| Paraben | | | | |
| Propylene | 4.4 | 4.4 | 4.4 | 4.4 |
| Glycol | | | | |
| BHA | 0.025 | 0.025 | 0.025 | 0.025 |
| NaOH | 0.32 | 3.2 | 0.32 | 0.32 |
| 25% w/w | | | | |
| solution | | | | |
| (q.s.) | | | | |

Sepineo P600 includes about 30-40% w/w acrylamide/sodium acryloyldimethyl taurate copolymer (e.g., with an ultra-high molecular weight of at least 10M Da, e.g., for use as a thickening agent), about 5-10% w/w polysorbate 80, and about 20-25% w/w isohexadecane (a C₁₆ alkane, i.e. 2-methylpentadecane, e.g., for use as a penetration enhancing solvent). Thus, certain formulations herein may include Sepineo P600, which results in the formulations including about 0.6-0.8% w/w acrylamide/sodium acryloyldimethyl taurate copolymer, about 0.1-0.2% w/w polysorbate 80 (which would be in addition to any polysorbate 80 already included in certain examples of formulations herein, e.g., formulation 5), and about 0.4-0.5% w/w isohexadecane. In some embodiments, certain formulations herein may include Sepineo P600, which results in the formulations including about 1.2-1.6% w/w acrylamide/sodium acryloyldimethyl taurate copolymer, about 0.2-0.4% w/w polysorbate 80 (which would be in addition to any polysorbate 80 already included in certain examples of formulations herein), and about 0.8-1.0% w/w isohexadecane. Without being bound by theory, the acrylamide/sodium acryloyldimethyl taurate copolymer is unlikely to be permeation enhancing based on its MW being about 10M Da or higher. Furthermore, additional polysorbate 80 from Sepineo P600 over that already in certain formulations would likewise be unlikely to contribute to permeation enhancing effects. Accordingly, without wishing to be bound by theory, isohexadecane may be a key ingredient in the formulations herein.

### Methods

The formulations described herein are useful in delivering therapeutically relevant amounts of one or more APIs through the skin of a subject to an underlying tissue, structure, or organ in the subject. Thus, provided herein are methods of delivering one or more APIs to a subject in need thereof, comprising topically administering an effective amount of a formulation described herein to the subject. In some embodiments, the topical administration is to a skin surface on an eyelid, not directly to a mucous membrane such as is the case for certain pilocarpine gel formulations. In some embodiments, the topical administration is to a skin surface located between the subject's upper eyelid eyelash and the corresponding eye's eyebrow. In some embodiments, the topical administration is to a skin surface at the superior papebral sulcus, to a skin surface at the inferior palpebral sulcus, or both. In some embodiments, the topical administration is to a skin surface at the superior eye lid crease, the inferior eye lid crease, the malar fold, the nasojugal fold, adjacent to the lateral canthus, or a combination thereof.

Also provided herein are methods of treating one or more eye diseases by delivering therapeutically relevant amounts of one or more active pharmaceutical agents through the skin, e.g., an eyelid, of a subject suffering from one or more eye diseases. Accordingly, in some embodiments, provided herein are methods of treating an eye disease in a subject in need thereof, comprising topically administering a therapeutically effective amount of a formulation provided herein to the subject. In some embodiments, the eye diseases treated include one or more of age-related macular degeneration, allergic conjunctivitis, blepharitis, chorioretinitis, diabetic macular edema, diabetic retinopathy, dry eye disease, episcleritis, geographic atrophy, glaucoma, graft versus host disease, inflammation due to gene therapy vectors, injury-related ocular inflammation or dry eye syndrome, iritis, keratitis, keratoconjunctivitis sicca, macular degeneration (wet or dry), meibomian gland dysfunction, myopia, non-infectious uveitis, ocular hyperemia, presbyopia, primary or secondary Sjögren's syndrome, redness, retinal inflammation, retinal vein occlusion, sterile conjunctivitis, Thygeson superficial punctate keratitis, or uveitis.

In some embodiments, described herein are methods, comprising administering a composition described herein to a subject.

In some embodiments, described herein are methods of treating a disease in a subject in need thereof, comprising topically administering a therapeutically effective amount of a composition described herein to the subject, wherein the composition includes at least one active pharmaceutical ingredient.

In some embodiments, described herein are methods, comprising administering Formulation 1, 2, 3, 4, 5, 6, 7, 8, 9, 10, 11, 12, 13, or 14 described herein to a subject.

In some embodiments, described herein are methods of treating a disease in a subject in need thereof, comprising topically administering a therapeutically effective amount of Formulation 1, 2, 3, 4, 5, 6, 7, 8, 9, 10, 11, 12, 13, or 14 described herein to the subject, wherein the composition includes at least one active pharmaceutical ingredient.

In some embodiments of the methods described herein, the disease is an eye disease.

In some embodiments of the methods described herein, the active pharmaceutical ingredient is delivered to skin of the subject or delivered to an internal tissue of the subject through skin of the subject. In some embodiments of the methods described herein, the active pharmaceutical ingredient is travoprost, pilocarpine (as the free base), pilocarpine nitrate, or pilocarpine hydrochloride.

In some embodiments of the methods described herein, the formulation is Formulation 3 and includes at least one API. In some embodiments of the methods described herein, the formulation is Formulation 3 and includes travoprost.

### Kits

In some embodiments, provided herein are packaged formulations, comprising a container holding a therapeutically effective amount of at least one formulation described herein, and instructions for using the at least one formulation in accordance with one or more of the methods provided herein.

The present formulations and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (e.g., Irgacure 184, 2959, e.g., 144-(2-Hydroxyethoxy)-phenyl]-2-hydroxy-2-methyl-1-propane-1-one), preferably water-soluble initiators (e.g. Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (e.g., by the addition of specific product information leaflets) into suitable containers (boxes, etc.).

According to further embodiments, the present formulations can also be provided in kit form combined with other components necessary for administration of the formulation to the patient. For example, disclosed kits, such as for use in the treatment of cancer, can further comprise, for example, administration materials including spatulas, rulers, and the like.

The kits are designed in various forms based on the specific deficiencies they are designed to treat.

The formulations provided herein may be prepared and placed in a container for storage at ambient or elevated temperature. When the formulations are stored in a polyolefin plastic container as compared to a polyvinyl chloride plastic container, discoloration of the formulations may be reduced. Without wishing to be bound by theory, the container may reduce exposure of the container's contents to electromagnetic radiation, whether visible light (e.g., having a wavelength of about 380-780 nm) or ultraviolet (UV) light (e.g., having a wavelength of about 190-320 nm (UV B light) or about 320-380 nm (UV A light)). Some containers also include the capacity to reduce adherence or adsorption of the active agent to the surface of the container. Some containers also include the capacity to reduce exposure of the container's contents to infrared light, or a second component with such a capacity. The containers that may be used include those made from a polyolefin such as polyethylene, polypropylene, polyethylene terephthalate, polycarbonate, polymethyl pentene, polybutene, or a combination thereof, especially polyethylene, polypropylene, or a combination thereof. In some embodiments, the container is a glass container, and the user scoops the contents out of the container with a measured spatula, scoop, or the like. In some embodiments the container is malleable, which permits the user to squeeze the contents out of the container. The container may further be disposed within a second container, for example, a paper, cardboard, paperboard, metallic film, or foil, or a combination thereof, container to further reduce exposure of the container's contents to UV, visible, or infrared light. The formulations provided herein benefit from reduced discoloration, decomposition, or both during storage in such containers. The formulations provided herein may need storage lasting up to, or longer than, three months; in some cases up to, or longer than, one year. The containers may be in any form suitable to contain the contents; for example, a bag, a bottle, a tube, or a box.

The following examples further illustrate aspects of the present disclosure. However, they are in no way a limitation of the teachings or disclosure as described herein.

### EXAMPLES

### Example 1: Effect of pH on Chemical/Physical Stability

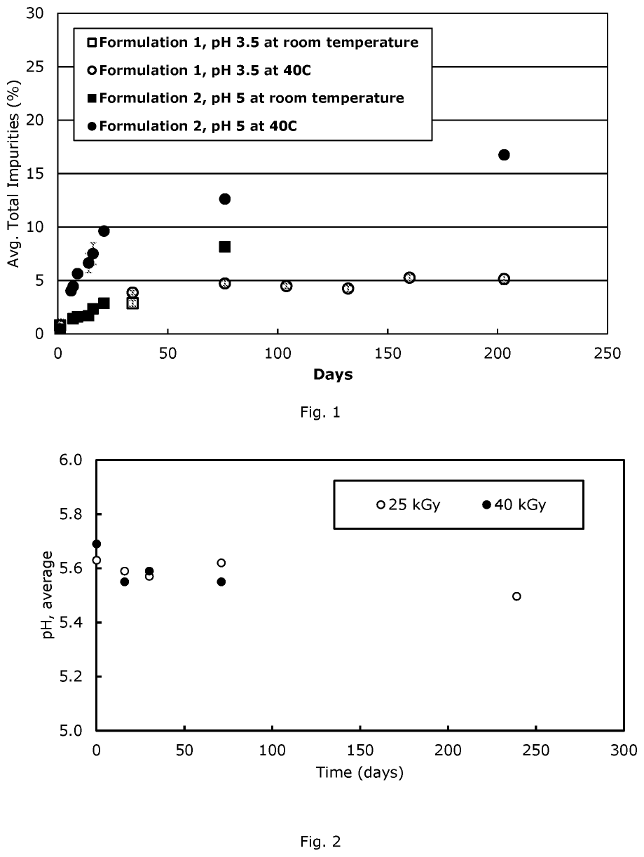

Oil-in-water formulations, including Formulation 1 (pH 3.5) and Formulation 2 (pH 5), were prepared and include pilocarpine (see Table 3 and Table 4 below). The formulations were compounded with/by the same compositions, equipment/processes and stored at controlled room temperature or 40° C. Oil-in-water Formulations 3-14 were similarly prepared. Samples were assessed by liquid chromatography over the course of multiple weeks for degradation impurities. Triplicate samples were used at each sampling time points. Results are shown in FIG. 1 and FIG. 7.

TABLE 3
Formulation 1, Formulation 2, Formulation 9, and Formulation 10.

| | Formulation | Formulation | Formulation | Formulation |
|---|---|---|---|---|
| | 1 | 2 | 9 | 10 |
| Ingredients | (% w/w) | (% w/w) | (% w/w) | (% w/w) |
| Pilocarpine | 4 | 4 | 4 | 4 |
| HCl | | | | |
| Polyoxyl 35 | 5 | 5 | 5 | 5 |
| castor oil | | | | |
| Propylene | 4.4 | 4.4 | 4.4 | 4.4 |
| glycol | | | | |
| Cetyl alcohol | 0.5 | 0.5 | 0.5 | 0.5 |
| Polysorbate 80 | 2 | 2 | 2 | 2 |
| Mineral oil | 10 | 10 | 10 | 10 |
| Sorbitol | 6 | 6 | 6 | 6 |
| PEG 8000 | 2 | 2 | 2 | 2 |
| Methyl | 0.18 | 0.18 | 0.18 | 0.18 |
| paraben | | | | |
| Propyl | 0.02 | 0.02 | 0.02 | 0.02 |
| paraben | | | | |
| Caffeine | 0.16 | 0.16 | 0.16 | 0.16 |
| Disodium | 0.2 | 0.2 | 0.2 | 0.2 |
| edetate | | | | |
| Carbomer | 4 | 4 | 4 | 4 |
| Homopolymer | | | | |
| Type C | | | | |
| Sepineo P600 | 0 | 0 | 4 | 4 |
| (comprising | | | | |
| 20-25% | | | | |
| isohexadecane) | | | | |
| Sodium | Q.S to pH | Q.S to pH | Q.S to pH | Q.S to pH |
| hydroxide | 3.5 | 5.0 | 3.5 | 5.0 |
| Purified water | Q.S to 100 | Q.S to 100 | Q.S to 100 | Q.S to 100 |

TABLE 4
Formulation 11, Formulation 12, Formulation
13, and Formulation 14.

| | Formulation | Formulation | Formulation | Formulation |
|---|---|---|---|---|
| | 11 | 12 | 13 | 14 |
| Ingredients | (% w/w) | (% w/w) | (% w/w) | (% w/w) |
| Pilocarpine or | 1 | 1.25 | 1.5 | 2 |
| its salt | | | | |
| Polyoxyl 35 | 5 | 5 | 5 | 5 |
| castor oil | | | | |
| Propylene | 4.4 | 4.4 | 4.4 | 4.4 |
| glycol | | | | |
| Cetyl alcohol | 0.5 | 0.5 | 0.5 | 0.5 |
| Polysorbate 80 | 2 | 2 | 2 | 2 |
| Mineral oil | 10 | 10 | 10 | 10 |
| Sorbitol | 6 | 6 | 6 | 6 |
| PEG 8000 | 2 | 2 | 2 | 2 |
| Methyl paraben | 0.18 | 0.18 | 0.18 | 0.18 |
| Propyl paraben | 0.02 | 0.02 | 0.02 | 0.02 |
| Caffeine | 0.16 | 0.16 | 0.16 | 0.16 |
| Disodium | 0.2 | 0.2 | 0.2 | 0.2 |
| edetate | | | | |
| Carbomer | 4 | 4 | 4 | 4 |
| Homopolymer | | | | |
| Type C | | | | |
| Sepineo P600 | 0 | 0 | 0 | 0 |
| (comprising | | | | |
| 20-25% | | | | |
| isohexadecane) | | | | |
| Sodium | Q.S to pH | Q.S to pH | Q.S to pH | Q.S to pH |
| hydroxide | 3.5 | 3.5 | 3.5 | 3.5 |
| Purified water | Q.S to 100 | Q.S to 100 | Q.S to 100 | Q.S to 100 |

FIG. 1 demonstrates the chemical stability fluctuations of pilocarpine in formulations having different pH levels. At controlled room temperature (about 20 to about 25° C.), the total impurities level of the pH 5 formulation was about 50% more than the pH 3.5 formulation. At 40° C., the total impurities level of the pH 5 formulation was over 100% more than the pH 3.5 formulation. Notably, at the two-month time point, the total impurities level of the pH 5 formulation stored at controlled room temperature exceeded that of the pH 3.5 formulation stored at 40° C. Furthermore, the total impurities level of the pH 3.5 formulation stored at 40° C. remained surprisingly stable, with respect to degradation of pilocarpine, for at least 3-6 months. That is, no further degradation of pilocarpine was observed after about 3 months; about 95% of the original pilocarpine remained intact after accelerated storage conditions of 40° C. for about 6 months (e.g., about 200 days), suggesting storage of the formulation achieved commercially relevant shelf-stability outcomes. Comparison of Formulation 9 and Formulation 10 also showed the benefit of pH 3.5 pilocarpine formulations provided herein over pH 5 pilocarpine formulations.

### Example 2: pH Stability Assay

Formulation 5 (comprising Formulation 3 and 0.004% w/w travoprost) was subjected to 40° C./75% RH storage condition and 40 kGy eBeam dose. Results are shown in FIG. 2. As can be seen, the formulation's pH was stable for at least about 8 months.

### Example 3: Viscosity Stability Assay

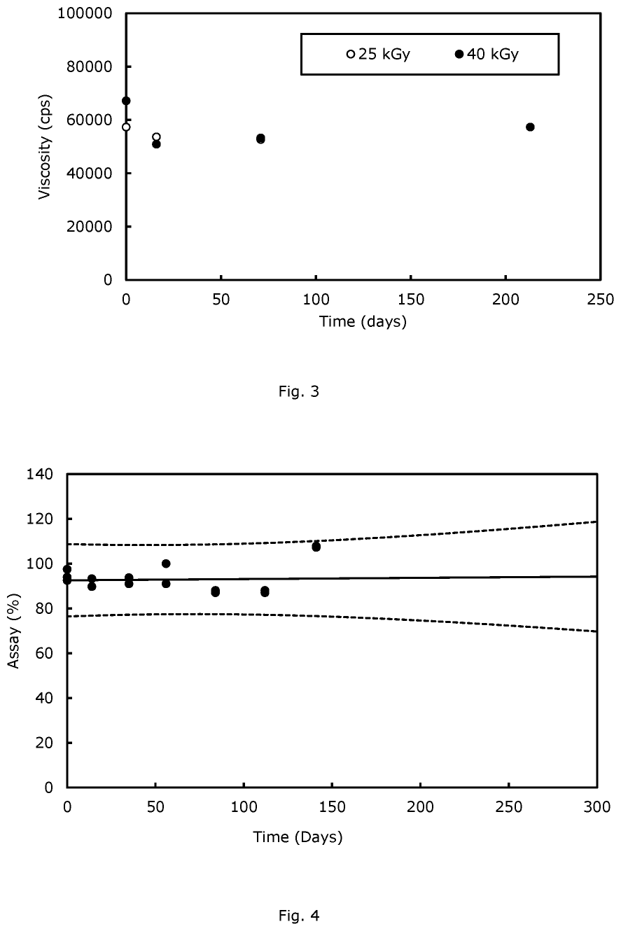

Formulation 5 was subjected to 40° C./75% RH storage condition and 40 kGy eBeam dose. Results are shown in FIG. 3. As can be seen, the formulation's viscosity was stable for at least about 7 months.

### Example 4: Sterilization Processing Stability Assay

Individual excipients or active pharmaceutical ingredients react differently to electron beam irradiation. Thus, it is important to verify that the maximum administered dose of irradiation during sterilization of a packaged product will not have a detrimental effect on the product's function or the patient's safety over the product's intended shelf life. Experimental samples of the product should be irradiated to at least the highest dose to be encountered during routine processing. For example, a product which is to receive an electron beam sterilizing dosage of 25 to 40 kiloGray (kGy) should be tested by dosing samples to at least 40 kGy. A conservative approach is to irradiate samples at doses up to twice the anticipated maximum dose.

Accordingly, Formulation 5 was subjected to 40° C./75% RH storage condition and 40 kGy eBeam dose. Travoprost concentration and impurities were measured to determine the stability profiles before and after the sterilization. Results are shown in FIG. 4. As can be seen, the formulation was physically stable for at least about 5 months, and projected to remain stable for at least one year.

### Example 5: Preclinical Studies of Formulation Usage

A formulation described herein is provided to a subject in a capped malleable plastic tube for use in treating an eye disease in the subject.

The length of the tip of the tube is used as a dosage size reference to dispense cream as a strip form. The cream is applied to an upper eyelid, a lower eyelid, or both, on one or both eyes of the subject. Care is taken by the subject to prevent the tip of the tube from touching the eye, hands or any other surfaces to keep it free from contamination. The subject refrains from wearing contact lenses or using artificial tears/ocular lubricants, and forgoes the use of eye makeup, including but not limited to eye shadow, eye creams/lotions/gels/serums, eyelash extensions, false eyelashes, or other eye products during the study. The subject also refrains from direct sunlight for 30 minutes after application of the cream. It is recommended to wear sunglasses that block ultraviolet A and B (UVA and UVB) light when outside in direct sunlight following application of the formulation.

Application sequence: 1. The subject washes their hands prior to applying the product around the eyes. 2. Break the tamper-evident seal and remove the cap from the tube. Do not use if the seal was broken before use. 3. Squeeze a quarter inch (approximately the same size as the length of the tip of the tube) of the ophthalmic topical cream from the tube in a straight line onto the fingertip. 4. To apply the cream to upper and/or lower eyelids, gently apply (avoid dragging or rubbing) the cream back and forth across the lower part of the upper eyelid until the cream is fully applied. Avoid placing the cream directly from the tube to the eyelid, applying too close to your eyelashes or in the eye. 5. Repeat steps 3 and 4 for the other eye, if present. 6. Place the cap securely back on the tube and store at room temperature. 7. The subject washes their hands after the product has been applied to both eyes. 8. The ophthalmic topical cream is applied on the upper eyelids of both eyes every 12 hours (e.g., BID in the morning (8 AM ±2 hours) and in the evening (8 PM ±2 hours). Following this application sequence, the formulation achieves therapeutically relevant bioavailability. Put another way, the subject's eye disease is treated to a statistically significant degree or by the subject's subjective observation of a reduction in one or more symptoms of the eye disease.

### Example 6: In Vivo Pharmacokinetic Dermal Topical Eyelid Administrative Study

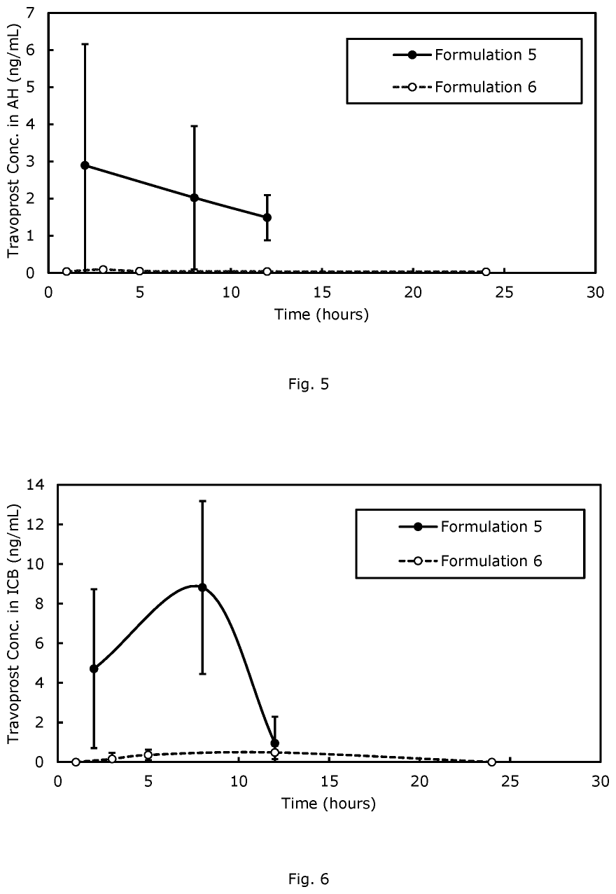

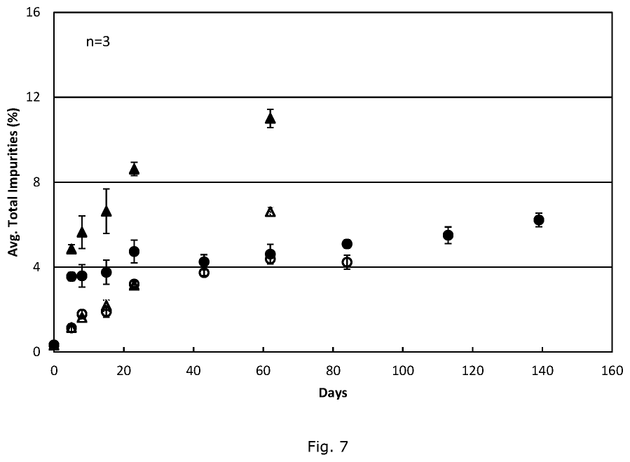

Formulation 5 (comprising Formulation 3 and 0.012% w/w travoprost) or Formulation 6 (comprising Formulation 4 and 0.012% w/w travoprost) is applied to eyelids in a minipig model. The aqueous humor (AH) and iris/ciliary body (ICB) are collected and analyzed by a liquid chromatography/tandem mass spectrometry (LC/MS-MS) method to quantitatively determine the model drug travoprost concentrations. The model drug travoprost concentration in AH and ICB after a single topical application of Formulation 5 and Formulation 6 are shown in FIG. 5 and FIG. 6.

The test results indicate that the model drug concentrations in AH and ICB are significantly and surprisingly higher with the Formulation 3-based formulation (e.g., Formulation 5) than those with the Formulation 4-based formulation (e.g., Formulation 6). By calculation, the average drug concentration in AH using Formulation 5 is approximately 48 folds higher than using Formulation 6 at 2-hour time point after application, while in ICB using Formulation 5 is approximately 20 folds higher than using Formulation 6 at 8-hour time point after application.

Fick's first law relates the diffusive flux to the gradient of the concentration. It postulates that the flux goes from regions of high concentration to regions of low concentration, with a magnitude that is proportional to the concentration gradient (spatial derivative), or in simplistic terms the concept that a solute will move from a region of high concentration to a region of low concentration across a concentration gradient. A diffusion process that obeys Fick's law is called normal or Fickian diffusion. The addition of 0.4-0.5% of isohexadecane to the formulations described herein alone would add an additional 0.3× (or 30%) of penetration by calculation (assuming a linear correlation between concentration and penetration from Fick's law of diffusion). But this factor alone cannot explain the greater than 20× of travoprost penetration increase achieved for Formulation 5 when compared with that of Formulation 6. Here, a particular combination of elements has been discovered that, when in a topical formulation, cause delivery of an API in the formulation to disobey Fick's law. Put another way, formulations that do not obey Fick's law have been discovered. The formulations display non-Fickian diffusion, or anomalous diffusion. Without wishing to be bound by theory, this may be explained by the unexpected or surprisingly synergistic effects among the excipients including the chemical permeation enhancer isohexadecane and the excipients EDTA and citrate.

While the disclosure has been described in detail and with reference to specific embodiments thereof, it will be apparent to one skilled in the art that various changes and modifications can be made without departing from the spirit and scope of the disclosure.

Groupings of alternative elements or embodiments disclosed herein may be referred to and claimed individually or in any combination with other members of the group or other elements found herein.

Unless otherwise defined, all technical and scientific terms used herein are accorded the meaning commonly known to one with ordinary skill in the art.

Those skilled in the art will recognize, or be able to ascertain, using no more than routine experimentation, many equivalents of the specific embodiments of the invention described herein. Such equivalents are intended to be encompassed by the following claims.

1. A composition, comprising about 0.05% to about 3% w/w C₇₋₂₀ alkane, about 0.005% to about 0.5% w/w metal chelating agent, and about 1 mM to about 500 mM buffer, and optionally including one or more of the following:
a. at least one antioxidant;
b. at least one base;
c. at least one methylxanthine;
d. at least one diol;
e. at least one emulsifier;
f. at least one Lewis acid;
g. at least one oil;
h. at least one preservative;
i. at least one thickening agent;
j. at least one tonicity agent;
k. water; or
I. at least one active pharmaceutical ingredient.

2. A composition, including:
zero or up to about 0.16% w/w caffeine;
about 0.5 to about 4% w/w carbomer homopolymer type C;
about 0.5% w/w cetyl alcohol;
about 0.02 to about 0.2% w/w disodium EDTA;
about 0.05 to about 0.18 methyl paraben;
about 10% w/w mineral oil;
about 2% PEG 8000;
about 5% w/w polyoxyl 35 castor oil;
about 2% w/w of a first polysorbate 80;
about 0.01 to about 0.02 propyl paraben;
about 4.4% w/w propylene glycol;
zero or up to about 4% w/w of a component, the component comprising about 30-40% w/w acrylamide/sodium acryloyldimethyl taurate copolymer, about 5-10% w/w of a second polysorbate 80, and about 20-25% w/w isohexadecane;
about 6% w/w sorbitol;
about 0.004 to about 4% w/w of at least one active pharmaceutical ingredient; and
a pH of about 3.5±0.5 or about 5.5±0.5;
wherein the composition optionally includes about 0.025% w/w butylated hydroxyanisole, and about 6 mM to about 7 mM potassium citrate.

3. The composition of claim 1, which is a cream formulation.

4. The composition of claim 1, which is an oil-in-water cream.

5. The composition of claim 1, wherein the C₇₋₂₀ alkane is an emollient.

6. The composition of claim 1, wherein the C₇₋₂₀ alkane is isohexadecane.

7. The composition of claim 5, including the at least one active pharmaceutical ingredient, which is travoprost.

8. The composition of claim 1, wherein the metal chelating agent is an ethylenediaminetetraacetic acid or a salt thereof.

9. The composition of claim 1, wherein the buffer is a citric acid or a salt thereof.

10. The composition of claim 1, including the at least one active pharmaceutical ingredient, which is selected from aflibercept, apraclonidine, atropine, betaxolol, brimonidine, brinzolamide, carbachol, dexamethasone, dipivefrin, donepezil, dorzolamide, levobunalol, metipranolol, pilocarpine, physostigmine, a prostaglandin, a steroid, or timolol, or a pharmaceutically acceptable salt thereof.

11. The composition of claim 1, including the at least one active pharmaceutical ingredient, which is selected from:
a prostaglandin selected from bimatoprost, latanoprost, prostaglandin F2a as an isopropyl ester, tafluprost, or travoprost, or their corresponding free acid; or
a steroid selected from an androgen, a corticosteroid, an estrogen, or a progestogen.

12. The composition of claim 1, including the at least one active pharmaceutical ingredient, which is selected from a glucocorticoid or a mineralocorticoid.

13. The composition of claim 1, including the at least one active pharmaceutical ingredient, which is loteprednol etabonate.

14. The composition of claim 1, wherein the C₇₋₂₀ alkane is isohexadecane, the metal chelating agent is ethylenediaminetetraacetic acid disodium salt, and the buffer is potassium citrate.

15. The composition of claim 13, including the at least one active pharmaceutical ingredient, which is travoprost.

16. The composition of claim 13, comprising about 0.4-0.5% w/w isohexadecane, and about 0.012% w/w travoprost.

17. The composition of claim 13, comprising about 6-7 mM potassium citrate, about 0.2% w/w ethylenediaminetetraacetic acid disodium salt, about 0.4-0.5% w/w isohexadecane, and about 0.012% w/w travoprost.

18. The composition of claim 1, wherein the composition comprises a pH of about 3.5±0.5.

19. The composition of claim 18, wherein the at least one active pharmaceutical ingredient includes pilocarpine or a pharmaceutically acceptable salt thereof.

20. The composition of claim 1, wherein the composition comprises a pH of about 5.5±0.5.

21. The composition of claim 20, wherein the at least one active pharmaceutical ingredient includes travoprost.

22. A method, comprising administration of the composition of claim 1 to a subject.

23. A method of treating a disease in a subject in need thereof, comprising topical administration of a therapeutically effective amount of the composition of claim 1 to the subject, wherein the composition includes the at least one active pharmaceutical ingredient.

24. The method of claim 23, wherein the disease is an eye disease.

25. The method of claim 23, wherein the active pharmaceutical ingredient is delivered to skin of the subject or delivered to an internal tissue of the subject through skin of the subject by the topical administration.
